(19) United States
(12) Patent Application Publication
(10) Pub. No.: US 2011/0264221 A1
(43) Pub. Date: Oct. 27, 2011

(54) Interspinous Fusion Device and Method

(75) Inventors: H. Randal Woodward, Omaha, NE (US); Mahmoud F. Abdelgany, Rockaway, NJ (US); Ahmad Faizan, Mountain Lakes, NJ (US)

(73) Assignee: Custom Spine, Inc., Parsippany, NJ (US)

(21) Appl. No.: 12/766,864

(22) Filed: Apr. 24, 2010

(51) Int. Cl. A61F 2/44; B23P 11/00

(52) U.S. Cl. 623/17.16; 29/525.11

(57) ABSTRACT

An interspinous fusion assembly includes a screw including a first end that includes at least one aperture, a second end inserted between the two spinous processes, a shank separating the first end from the second end. The shank includes an outer surface that includes cutting means and at least one hole bored through the surface. An inner chamber positioned in the shank and substantially along a longitudinal axis of the shank. The inner chamber includes the aperture of the first end and connects with the hole of the shank. A pair of complementary plates bilaterally positioned with respect to one another and accommodating the screw. A fastening mechanism positioned in one of the plates that retains a relative position of the pair of complementary plates constant with respect to one another. The pair of complementary plates includes a first plate and a second plate.

### BACKGROUND

1. Technical Field

The embodiments herein generally relate to medical devices, and, more particularly, to screw and plate systems for interspinous processes.

2. Description of the Related Art

The spinal column is a bio-mechanical structure composed primarily of ligaments, muscles, vertebrae and intervertebral disks. The spinal column functions as a support to the body, which involves the transfer of the weight, bending movements, and relatively complex physiological motion of the human body parts which may lead to spinal stenosis. Spinal stenosis is a medical condition that narrows the spinal canal and the foramina which compresses the enclosed neuro structures (nerves). This is usually due to the common occurrence of spinal degeneration that occurs with aging. It can also sometimes be caused by spinal disc herniation, a tumor, or occasionally a synovial cyst. Spinal stenosis may affect the cervical, thoracic, or lumbar spine. In some cases, it may be present in all three places in the same patient.

To overcome this, decompression and spinal fusion is performed by which two or more vertebrae or spinous processes are fused together with bone grafts and internal implants. This process immobilizes the vertebral segments, thus eliminating pain of the spine, but may create pressure on the spinal nerves. Accordingly, there remains a need to perform decompression to relieve pressure on the spinal nerves by distracting and fusing the adjacent spinous processes.

### SUMMARY

In view of the foregoing, an embodiment herein provides an interspinous fusion assembly that includes a screw including a first end that includes at least one aperture, a second end inserted between two spinous processes, a shank separating the first end from the second end. The shank includes an outer surface that includes cutting means and at least one hole bored through the surface. An inner chamber positioned in the shank and substantially along a longitudinal axis of the shank. The inner chamber includes the aperture of the first end and connects with the hole of the shank. A pair of complementary plates bilaterally positioned with respect to one another and accommodating the screw. A fastening mechanism positioned in one of the plates that retains a relative position of the pair of complementary plates constant with respect to one another.

The pair of complementary plates includes a first plate and a second plate. Each plate includes a first lateral side including a first arm, a second lateral side positioned opposite to the first lateral side. The second lateral side includes a second arm. The pair of complementary plates further includes a plurality of spikes outwardly projecting from the first arm and the second arm. The first plate includes a first projection positioned in between the first arm and the second arm, and a first slot positioned opposite to the first projection. The first plate further includes a plate surface that separates the first arm from the second arm. The first projection includes a first surface. The plate surface and the first surface are co-planar.

The first projection includes a first hole and a second hole. The first hole and the second hole are transversely positioned with respect to one another and intersect one another. The first hole accommodates the fastening mechanism. The second plate includes a second projection positioned in between the first arm and the second arm. The second projection includes an outwardly protruding knob. The second hole of the first plate accommodates the knob. The fastening mechanism engages the knob positioned in the second hole. The second plate includes a second slot positioned opposite to the second projection. The first slot and the second slot accommodate the screw.

In another aspect, an apparatus for stabilizing interspinous processes is provided. The apparatus includes an interspinous process screw positioned between the interspinous processes. The interspinous process screw includes a first end that includes an opening, a second end including a tip, a shank separating the first circular end from the second end, a plurality of cutting mechanisms on the shank, a plurality of holes bored through the shank, and a chamber configured through the shank and terminating at the opening of the first end. The chamber is transversely positioned with respect to the plurality of holes.

A first plate operatively connected to the first end of the interspinous process screw. The first plate includes a first pair of arms, a first slot, and a first projection that includes a first hole and a second hole. A second plate operatively connected to the second end of the interspinous process screw. The second plate includes a second pair of arms, a second slot, and a second projection that includes an outwardly protruding knob that engages the second hole. The apparatus further includes a set screw that engages the first hole and the knob. The first slot accommodates the first end and the shank of the interspinous process screw. The second slot accommodates the second end and the shank of the interspinous process screw.

The first plate includes a plate surface positioned between the first pair of arms. The first projection includes a first surface. The plate surface and the first surface care co-planar. The chamber accommodates bone graft material. The plurality of holes in the shank accommodate bone graft material. Each of the first plate and the second plate include a plurality of outwardly projecting spikes that attach to the interspinous processes.

In yet another aspect, a method of assembling an interspinous fusion assembly between two interspinous processes is provided. The assembly includes a screw that includes oppositely positioned ends separated by a shank that includes a surface including cutting means and at least one hole bored through the surface and terminating at an inner chamber positioned in the shank and connecting with the at least one hole. The assembly further includes a pair of complementary plates bilaterally positioned with respect to one another and accommodating the screw. The pair of plates includes a first plate and a second plate, and a fastening mechanism positioned in one of the plates.

The method includes inserting the screw between the two interspinous processes, attaching the first plate to the interspinous process screw, and attaching the second plate to the interspinous process screw. The first plate further includes a first hole and a second hole. The second plate further includes an outwardly protruding knob. The knob of the second plate is inserted into the second hole of the first plate. The fastening mechanism is engaged in the first hole of the first plate to lock the knob of the second plate to the first plate. The cutting means anchor to bone.

At least one hole that receives bone graft material. The inner chamber accommodates bone graft material and accepts a surgical instrument used to drive the screw between the spinous processes. Each of the first plate and the second plate include a pair of oppositely positioned arms that include a plurality of spikes outwardly protruding from the arms. The plurality of spikes attach to the interspinous processes. The first plate includes a first projection that includes a first surface, a plate surface positioned between the arms of the first plate. The first surface and the plate surface are co-planar. The fastening mechanism retains a relative position of the pair of complementary plates constant with respect to one another.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1 illustrates a perspective view of an interspinous fusion assembly according to an embodiment herein;

FIG. 2A illustrates a perspective view of an interspinous process screw of the assembly of FIG. 1 according to an embodiment herein;

FIG. 2B illustrates a cross-sectional view of the interspinous process screw of FIG. 2A according to an embodiment herein;

FIG. 3A illustrates a perspective view of a first plate of the assembly of FIG. 1 according to an embodiment herein;

FIG. 3B illustrates a perspective view of a second plate of the assembly of FIG. 1 according to an embodiment herein;

FIG. 4 illustrates a perspective view of a set screw of the assembly of FIG. 1 according to an embodiment herein;

FIG. 5 illustrates the assembly of FIG. 1 fitted between two spinous processes according to a first embodiment herein;

FIG. 6 illustrates the assembly of FIG. 1 fitted between two spinous processes according to a second embodiment herein; and

FIG. 7 is a flow diagram illustrating a method of assembling a dynamic screw and plate system according to an embodiment herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

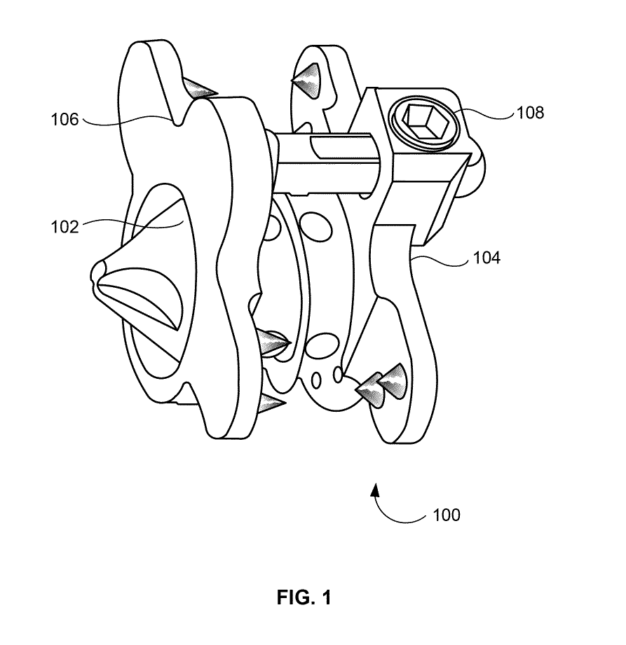

As mentioned, there remains a need to perform indirect decompression to relieve pressure on the spinal nerves by distracting and fusing the adjacent spinous processes. The embodiments herein achieve this by providing an interspinous fusion assembly. Referring now to the drawings, and more particularly to FIGS. 1 through 7, where similar reference characters denote corresponding features consistently throughout the figures, there are shown preferred embodiments.

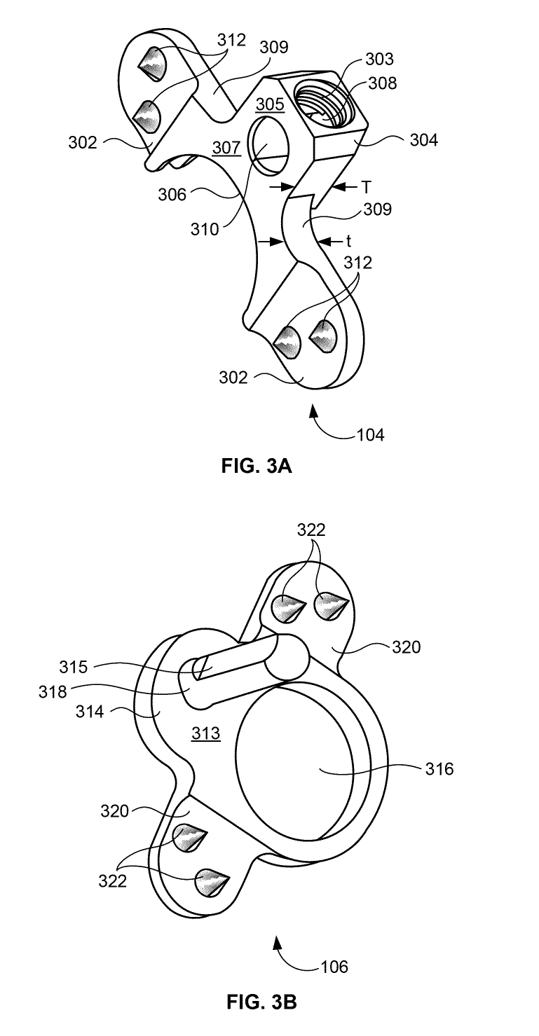

FIG. 1 illustrates a perspective view of an interspinous fusion assembly 100 having an interspinous process screw 102, a first plate 104, a second plate 106, and a set screw 108 according to an embodiment herein. The interspinous process screw 102 is characterized by a relatively large thread pitch (not shown in FIG. 1). The interspinous process screw 102 is placed laterally between the interspinous processes (not shown in FIG. 1). The first plate 104 and the second plate 106 are placed in a bilateral direction for assembling the interspinous process screw 102. The set screw 108 fits in a hole 310 (shown in FIG. 3A) of the first plate 104.

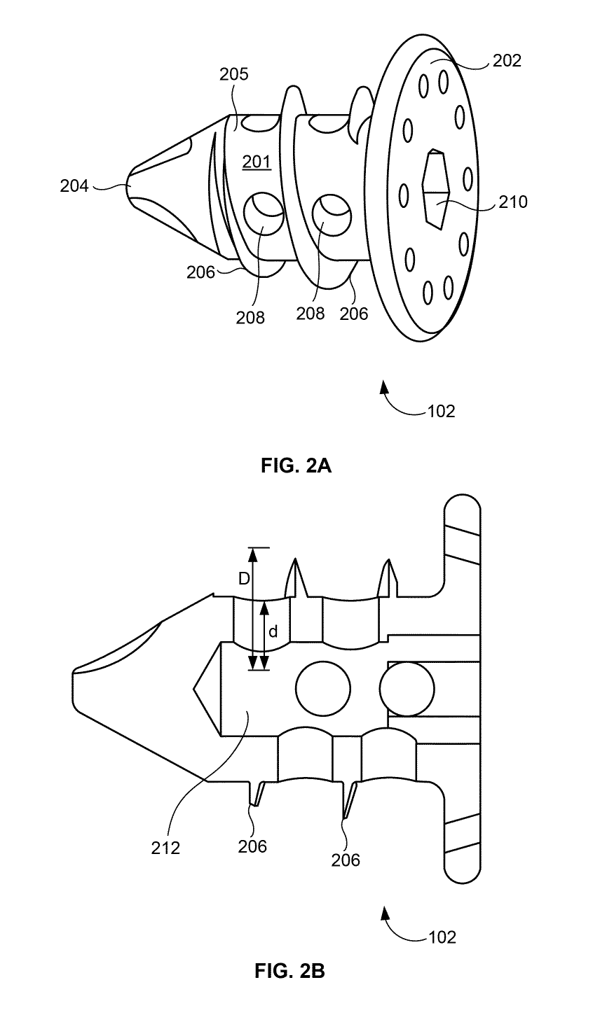

FIGS. 2A and 2B illustrate a perspective view and a cross-sectional view, respectively, of the interspinous process screw 102 of FIG. 1 according to an embodiment herein. The interspinous process screw 102 includes a first circular end 202, a second circular end 204, a plurality of cutting mechanism such as threads or flutes 206, a plurality of holes 208, a tool receiving aperture 210, and an open cylindrical inner chamber 212. The first circular end 202 includes the tool receiving aperture 210 that accepts a surgical instrument (or a screwdriver) for rotating the interspinous process screw 102, and while FIG. 2A illustrates a hexagonal configuration for the tool receiving aperture 210, those skilled in the art would understand that many different configurations may be used in accordance with the embodiments herein, and that the embodiments herein are not limited to one particular type of configuration. For example, the tool receiving aperture 210 may be any of a circular structure, a square structure, a pentagonal structure, etc. that accepts the surgical instrument for rotating the interspinous process screw 102. Alternatively, the aperture 210 may be closed at the first end 202 such that a raised nut (not shown) may be configured on the first end 202 to form a bolt-like device, which may be engaged by a corresponding respective tool (not shown). The first circular end 202 includes a plurality of holes that are optional securing anchor sutures and extend completely through the first circular end 202.

In one embodiment, the first circular end 202 is configured as a cylindrical flange structure. The second circular end 204 is positioned opposite to the first circular end 202 and, in one embodiment, is configured as a pointed conical structure with a circular tip. In one example embodiment, the interspinous process screw 102 has a major diameter D and a minor diameter d that are configured to have a relatively high ratio (i.e., D/d). In this embodiment, the high ratio permits a more secure abutment onto the adjacent spinous processes. The second circular end 204 includes the plurality of cutting threads or flutes 206 formed on the outer surface 201 of the shank 205, although the embodiments herein may include a non-threaded shank 205.

The plurality of cutting threads or flutes 206 are dimensioned and configured to anchor through bone. The plurality of holes 208 in the screw shank 205 allows a bone growth between the superior and inferior spinous processes. The open cylindrical chamber 212 is configured to accommodate bone graft material (not shown). Furthermore, the plurality of holes 208 are positioned transversely to the inner chamber 212 such that the longitudinal axis of each hole 208 intersects with the longitudinal axis of the inner chamber 212 and the corresponding tool receiving aperture 210.

FIG. 3A illustrates a perspective view of the first plate 104 of the assembly 100 FIG. 1 according to an embodiment herein. The first plate 104 includes a pair of flanges 309, a pair of arms 302, a first projection 304, a first slot 306, a first hole 308, a second hole 310, and a plurality of spikes 312. The first slot 306 is dimensioned and configured to accommodate the shank 205 of the interspinous process screw 102 of FIGS. 2A and 2B. The first and second holes 308, 310 are transversely positioned with respect to one another in the first projection 304 such that the longitudinal axis of hole 308 intersects with the longitudinal axis of hole 310.

In one embodiment, the first hole 308 comprises threads 303. Moreover, in one embodiment, the first plate 104 is symmetric such that each arm 302 is evenly spaced with respect to the generally central location of the first projection 304. Additionally, the first projection 304 comprises a first surface 305 that is planar with surface 307 that separates the pair of arms 302 from one another. Additionally, in one embodiment, the first projection 304 comprises a thickness T that is greater than the thickness t of the flanges 309 that connect to the pair of arms 302. The first slot 306 is configured to accommodate the first circular end 202 of interspinous process screw 102 of FIG. 1. The first hole 308 is dimensioned and configured to accommodate the set screw 108 of FIG. 1. The second hole 310 is configured and horizontally positioned to the first hole 308. The plurality of spikes 312 are configured to attach to the Interspinous processes.

FIG. 3B illustrates a perspective view of the second plate 106 of the assembly 100 of FIG. 1 according to an embodiment herein. The second plate 106 includes a pair of arms 320, a plurality of spikes 322, a second projection 314, a second slot 316, and an outwardly protruding knob 318. The second slot 316 is dimensioned and configured in a ring-like configuration to support the second circular end 204 and/or the shank 205 of the interspinous process screw 102. The plurality of spikes 312 are configured to attach to the interspinous processes. The outwardly protruding knob 318 extends from the surface 313 of the second projection 314. The outwardly protruding knob 318 is dimensioned and configured to engage the second hole 310 of the first plate 104 of FIG. 3A. The knob 318 may have a uniform cylindrical configuration or may have an offset and portion cut out of one or more sides of the knob 318 to define a cut out surface 315 as shown in FIG. 3B.

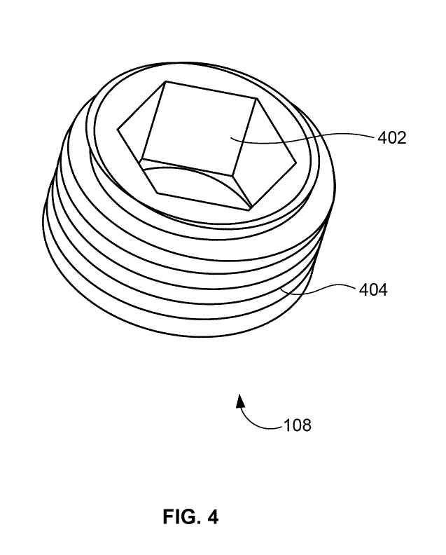

FIG. 4 illustrates a perspective view of the set screw 108 of the assembly 100 of FIG. 1 having an open head 402 and a threaded side 404 according to an embodiment herein. The set screw 108 acts as a fastening mechanism to retain the knob 318 within the second hole 310 in order to prevent the knob 318 from sliding out of the second hole 310, and thereby retaining the relative position of the first plate 104 to the second plate 106 constant once the set screw 108 is tightened in place. The open head 402 may be configured in any suitable configuration, and in one embodiment, as shown in FIG. 4, the open head 402 is configured as a hexagonal head 402, although other configurations are possible. The open head 402 may be tightened by using a screwdriver or other suitable tool (not shown).

The threaded side 404 is dimensioned and configured to engage the threads 303 of the first hole 308 of the first plate 104 of FIG. 3A. Although, those skilled in the art would appreciate that a non-threaded fastening mechanism such as a push type set screw could also be used in accordance with the embodiments herein, and in such an embodiment, the corresponding first hole 308 of the first plate 104 of FIG. 3A would be non-threaded.

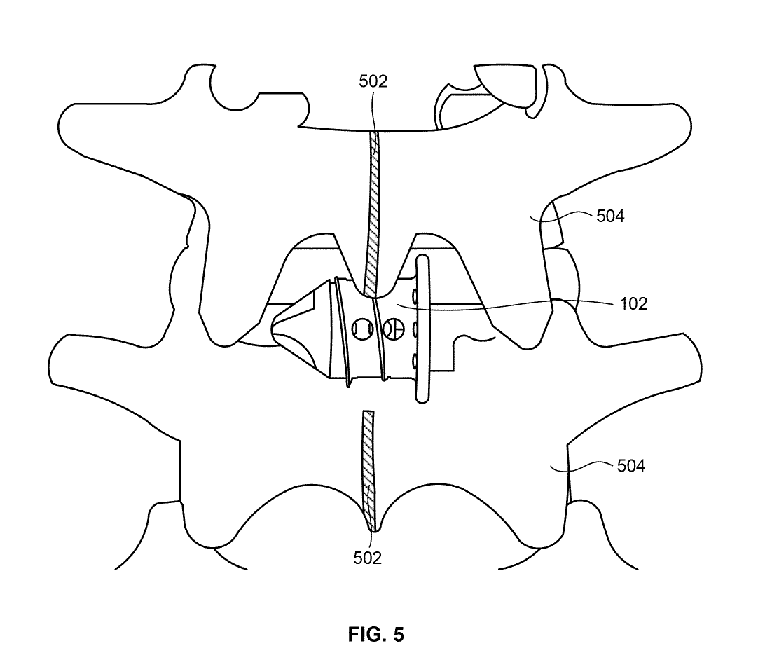

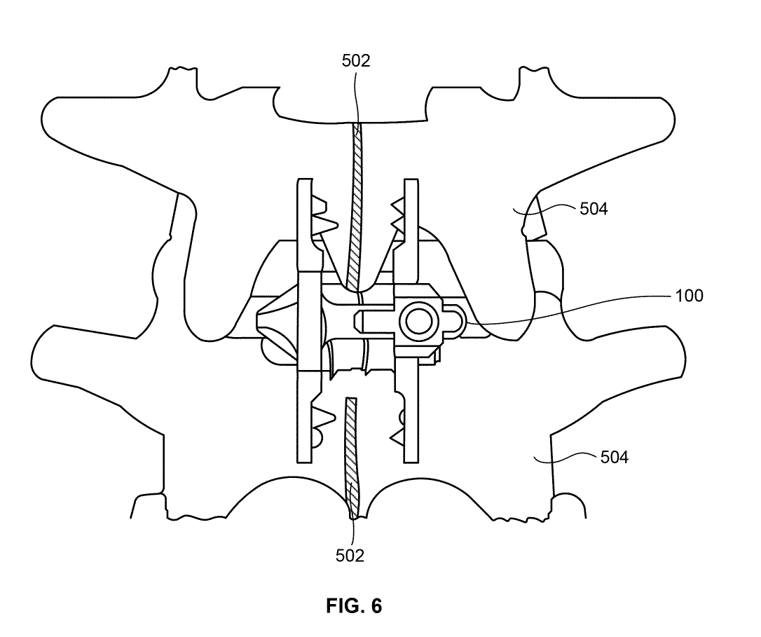

FIG. 5 illustrates an application of the interspinous process screw 102 of FIG. 1 inserted between two spinous processes 502 in pars interarticularis 504 according to a first embodiment herein. FIG. 6 illustrates an application of the assembly 100 of FIG. 1 inserted between two spinous processes 502 in the pars interarticularis 504 of FIG. 5 according to a second embodiment herein. The first plate 104 and the second plate 106 are placed bilaterally to assemble the entire construct assembly 100. Bone graft (not shown) is placed inside the open cylindrical chamber 212 of the interspinous process screw 102 of FIG. 1. The plurality of holes 208 on the shaft of the interspinous process screw allows bone growth between the spinous processes 502. The pressure on the spinal nerves is relieved by distracting and fusing the adjacent spinous processes.

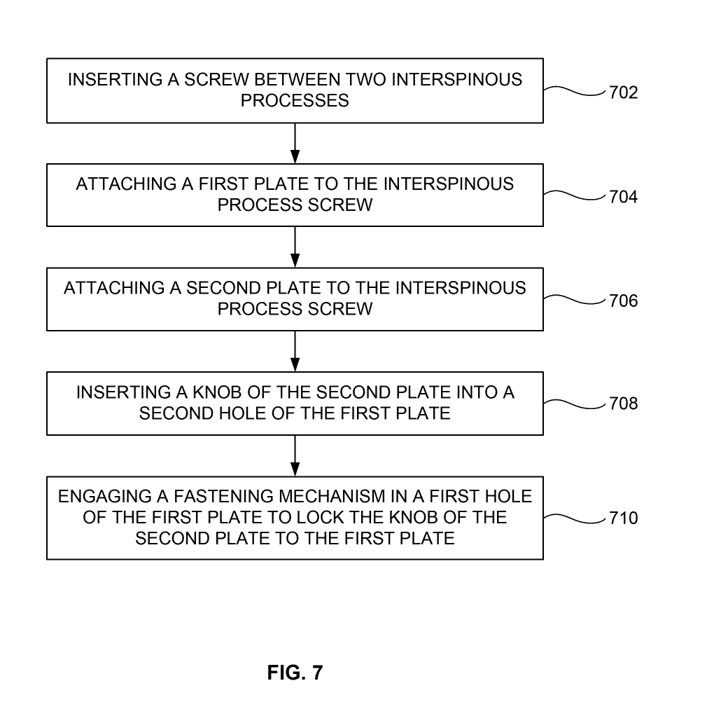

FIG. 7, with reference to FIGS. 1 through 6, is a flow diagram illustrating a method of assembling a dynamic screw and plate assembly 100 according to an embodiment herein. The assembly 100 includes a screw 102 that includes oppositely positioned ends 202, 204 separated by a shank 205 including a surface 201 that includes cutting means 206 and at least one hole bored through the surface 201 and terminating at an inner chamber 212 positioned in the shank 205 and connecting with at least one hole 208. The assembly 100 further includes a pair of complementary plates 104, 106 bilaterally positioned with respect to one another and accommodating the screw 102. The pair of plates includes a first plate 104 and a second plate 106. A fastening mechanism 108 is positioned in one of the plates 104.

In step 702, the screw 102 is inserted between the two interspinous processes 502. In step 704, the first plate 104 is attached to the interspinous process screw 102. The first plate 104 further includes a first hole 308 and a second hole 310. In step 706, the second plate 106 is attached to the interspinous process screw 102. The second plate 106 further includes an outwardly protruding knob 318. In step 708, the knob 318 of the second plate 106 is inserted into the second hole 310 of the first plate 104. In step 710, the fastening mechanism 108 is engaged in the first hole 308 of the first plate 104 to lock the knob 318 of the second plate 106 to the first plate 104.

The cutting means 206 anchor to bone and at least one hole 208 receives bone graft material (not shown). The inner chamber 212 accommodates bone graft material and accepts a surgical instrument (not shown) used to drive the screw 102 between the spinous processes 502. Each of the first plate 104 and the second plate 106 include a pair of oppositely positioned arms 302, 320 that includes a plurality of spikes 312, 322 outwardly protruding from the arms 302, 320. The plurality of spikes 312, 322 are attached to the interspinous processes 502. The first plate 104 includes a first projection 304 that includes a first surface 305, and a plate surface 307 positioned between the arms 302 of the first plate 104. The first surface 305 and the plate surface 307 are co-planar. The fastening mechanism 108 retains a relative position of the pair of complementary plates 104, 106 constant with respect to one another

The assembly 100 indirectly decompresses the spinal nerves by distraction and fusing the adjacent spinous processes of two or more vertebra. The assembly 100 immobilizes the functional spine unit. Moreover, the assembly 100 includes a plurality of holes 208 and an inner chamber 212, which allows the bone to grow between the spinous processes 502 of adjacent vertebrae.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. An interspinous fusion assembly comprising:
a screw comprising:
a first end comprising at least one aperture;
a second end inserted between two spinous processes;
a shank separating said first end from said second end, wherein said shank comprises an outer surface comprising cutting means and at least one hole bored through said surface; and
an inner chamber positioned in said shank and substantially along a longitudinal axis of said shank, wherein said inner chamber comprises said at least one aperture of said first end and connects with said at least one hole of said shank;
a pair of complementary plates bilaterally positioned with respect to one another and accommodating said screw; and
a fastening mechanism positioned in one of the plates, wherein said fastening mechanism retains a relative position of said pair of complementary plates constant with respect to one another.

2. The assembly of claim 1, wherein said pair of complementary plates comprise a first plate and a second plate, each plate comprising:
a first lateral side comprising a first arm; and
a second lateral side positioned opposite to said first lateral side, said second lateral side comprising a second arm; and
a plurality of spikes outwardly projecting from said first arm and said second arm.

3. The assembly of claim 2, wherein said first plate comprises:
a first projection positioned in between said first arm and said second arm; and
a first slot positioned opposite to said first projection.

4. The assembly of claim 3, wherein said first plate comprises a plate surface that separates said first arm from said second arm, wherein said first projection comprises a first surface, and wherein said plate surface and said first surface are co-planar.

5. The assembly of claim 3, wherein said first projection comprises a first hole and a second hole, wherein said first hole and said second hole are transversely positioned with respect to one another and intersect one another, and wherein said first hole accommodates said fastening mechanism.

6. The assembly of claim 5, wherein said second plate comprises a second projection positioned in between said first arm and said second arm.

7. The assembly of claim 6, wherein said second projection comprises an outwardly protruding knob, wherein said second hole of said first plate accommodates said knob, and wherein said fastening mechanism engages said knob positioned in said second hole.

8. The assembly of claim 6, wherein said second plate comprises a second slot positioned opposite to said second projection.

9. The assembly of claim 8, wherein said first slot and said second slot accommodate said screw.

10. An apparatus for stabilizing interspinous processes, said apparatus comprising:
an interspinous process screw positioned between said interspinous processes, said interspinous process screw comprising:
a first end comprising an opening;
a second end comprising a tip;
a shank separating said first circular end from said second end;
a plurality of cutting mechanisms on said shank;
a plurality of holes bored through said shank; and
a chamber configured through said shank and terminating at said opening of said first end, wherein said chamber is transversely positioned with respect to said plurality of holes;
a first plate operatively connected to said first end of said interspinous process screw, said first plate comprising:
a first pair of arms;
a first slot; and
a first projection comprising a first hole and a second hole;
a second plate operatively connected to said second end of said interspinous process screw, said second plate comprising:
a second pair of arms;
a second slot; and
a second projection comprising an outwardly protruding knob that engages said second hole; and
a set screw engaging said first hole and said knob.

11. The apparatus of claim 10, wherein said first slot accommodates said first end and said shank of said interspinous process screw, and wherein said second slot accommodates said second end and said shank of said interspinous process screw.

12. The apparatus of claim 10, wherein said first plate comprises a plate surface positioned between said first pair of arms, wherein said first projection comprises a first surface, and wherein said plate surface and said first surface are co-planar.

13. The apparatus of claim 10, wherein said chamber accommodates bone graft material, and wherein said plurality of holes in said shank accommodate bone graft material.

14. The apparatus of claim 10, wherein each of said first plate and said second plate comprise a plurality of outwardly projecting spikes that attach to said interspinous processes.

15. A method of assembling an interspinous fusion assembly between two interspinous processes, said assembly comprising a screw comprising oppositely positioned ends separated by a shank comprising a surface comprising cutting means and at least one hole bored through said surface and terminating at an inner chamber positioned in said shank and connecting with said at least one hole; a pair of complementary plates bilaterally positioned with respect to one another and accommodating said screw, wherein the pair of plates comprises a first plate and a second plate; and a fastening mechanism positioned in one of the plates, wherein said method comprises:
inserting said screw between said two interspinous processes;
attaching said first plate to said interspinous process screw, wherein said first plate further comprises a first hole and a second hole;
attaching said second plate to said interspinous process screw, wherein said second plate further comprises an outwardly protruding knob;
inserting said knob of said second plate into said second hole of said first plate; and
engaging said fastening mechanism in said first hole of said first plate to lock said knob of said second plate to said first plate.

16. The method of claim 15, wherein said cutting means anchor to bone, and wherein said at least one hole receives bone graft material.

17. The method of claim 15, wherein said inner chamber accommodates bone graft material and accepts a surgical instrument used to drive said screw between the spinous processes.

18. The method of claim 15, wherein said first plate and said second plate each comprise a pair of oppositely positioned arms comprising a plurality of spikes outwardly protruding from the arms, and wherein said plurality of spikes attach to said interspinous processes.

19. The method of claim 15, wherein said first plate comprises:
a first projection comprising a first surface; and
a plate surface positioned between said arms of said first plate,
wherein said first surface and said plate surface are co-planar.

20. The method of claim 15, wherein said fastening mechanism retains a relative position of said pair of complementary plates constant with respect to one another.
